(19) United States
(12) Patent Application Publication
(10) Pub. No.: US 2014/0323416 A1
(43) Pub. Date: Oct. 30, 2014

(54) 5-METHYLDIHYDROMORPHONE COMPOUNDS FOR TREATMENT OF PAIN

(71) Applicants: Anthony MARFAT, Mystic, CT (US); Peter CORNELIUS, Old Lyme, CT (US); Robert VOLKMANN, Mystic, CT (US); Panayiotis ZAGOURAS, Old Saybrook, CT (US); Frederick Raymond NELSON, Groton, CT (US); Anton Franz Joseph FLIRI, Stonington, CT (US)

(72) Inventors: Anthony MARFAT, Mystic, CT (US); Peter CORNELIUS, Old Lyme, CT (US); Robert VOLKMANN, Mystic, CT (US); Panayiotis ZAGOURAS, Old Saybrook, CT (US); Frederick Raymond NELSON, Groton, CT (US); Anton Franz Joseph FLIRI, Stonington, CT (US)

(21) Appl. No.: 14/254,711

(22) Filed: Apr. 16, 2014

Related U.S. Application Data
(60) Provisional application No. 61/813,142, filed on Apr. 17, 2013.

(51) Int. Cl. C07D 489/02; C07H 15/26; C07F 9/6561

(52) U.S. Cl. CPC C07D 489/02; C07F 9/6561; C07H 15/26
USPC 514/23; 546/23; 514/81; 546/45; 514/282; 536/18.7

(57) ABSTRACT

The aspects of the present disclosure are directed to novel compounds, formulations containing said compounds or pharmaceutically acceptable salts thereof which are suitable for administration to a patient. In particular, to 5-methyldihydromorphone prodrug compounds described in general Formula I and II and the use of the compounds for treating pain associated with a variety of chronic human disorders including for example neuropathic pain and pain associated with cancer, surgeries or injuries.

### FIELD OF THE INVENTION

The invention is directed to novel compounds, formulations containing said compounds or pharmaceutically acceptable salts thereof which are suitable for administration to a patient.

### BACKGROUND OF THE INVENTION

This invention is directed to 5-methyldihydromorphone product compounds described in general Formula I and II and the use of said compounds for treating pain associated with a variety of chronic human disorders including for example neuropathic pain and pain associated with cancer, surgeries or injuries. A second aspect of this invention pertains to a method comprising administering to a patient an effective amount of said 5-methyldihydromorphone pro-drug compounds of the invention wherein upon administration to the patient, release of the parent drug from the pro-drug is achieved in a controlled fashion.

The compounds of the invention may be generically categorized within the class of compounds known as opioids. It is well known that opioid drugs target three types of endogenous opioid receptors (i.e., mu, delta and kappa receptors) in biological systems. Most opioids, such as morphine, are mu opioid agonists that are often used as analgesics for the treatment of severe pain due to their activation of mu opioid receptors in the brain and central nervous system (CNS). Opioid receptors are, however, not limited to the CNS, and have been found in other tissues throughout the body. A number of side effects of opioid drugs may be caused by activation of these peripheral receptors. Opioids are also generally known to cause nausea and vomiting as well as inhibition of normal propulsive gastrointestinal function in animals and man (Reisine, T., and Pasternak, G., Goodman & Gilman's The Pharmacological Basis of Therapeutics Ninth Edition 1996, 521-555) resulting in side effects such as, for example, constipation. It has been reported that acute nausea or vomiting may occur in up to about 33% of patients who receive oral narcotic analgesics and in up to about 80% of patients who receive injectable narcotics following surgery or trauma. This is due, at least in part, to direct effects of narcotics on the gastrointestinal (GI) tract.

Prescription opioid use is also the subject of extensive abuse which is increasing and exacts a high toll on patients, physicians, and society. Nonmedical users of prescription pain relievers are perhaps the most troublesome population of individuals who abuse opioids; their number more than quadrupled from 1990 to 2000, with abuse of oxycodone and hydrocodone products particularly common. Escalating prescription drug abuse is associated with higher rates of comorbidities and drug-related mortality. The overall cost of prescription opioid abuse in the United States has been estimated at $9.5 billion, including health care, criminal justice, and workplace costs. Physicians who prescribe opioids must maintain extensive documentation and may be subject to investigation by the Drug Enforcement Administration.

Chronic pain and prescription opioid abuse are both highly prevalent. Chronic pain affects approximately 50 million Americans each year, whereas 48 million Americans 12 years or older have used prescription drugs for nonmedical reasons in their lifetimes. Among the most potent analgesics available, opioids have a recognized role in the treatment of cancer- and noncancer-related chronic pain conditions. Yet many physicians, concerned that their patients will become addicted, are reluctant to prescribe these agents, contributing to the widespread undertreatment of chronic pain. Physicians must realize that patients exhibit a continuum of behaviors in response to opioid therapy. In practice, prescription opioid users are in heterogeneous categories that include extreme cases of medical and nonmedical abusers. However, most patients who take prescription opioids are somewhere in between, ranging from those with pain who adhere to their treatment regimen to those who purposefully abuse their medications or from nonmedical users who self-medicate by taking illicit opioids to those who abuse opioids recreationally.

Opioid formulations that incorporate barriers to common forms of manipulation are an emerging component of risk management. Novel subclasses of opioid formulations, incorporating pharmacological strategies and physical barriers, are designed to deter or resist misuse and abuse by making it difficult to obtain euphoric effects from opioid use. To obtain euphoric effects, most individuals who abuse opioids crush the tablets or capsules and snort or inject them, increasing opioid bioavailability. In this way, a long-acting formulation can be made to release its full dose immediately. When extraction of the active drug is difficult because of the inclusion of physical barriers, the attractiveness of an opioid for abuse may be reduced. As pharmacologically proactive tools, these formulations use either pharmacodynamic or physical mechanisms to make opioids unattractive to individuals who abuse them, as well as present barriers to unintentional or deliberate misuse. However, the true ability of these formulations to reduce misuse and abuse will be unknown until they are approved, and widespread epidemiological data regarding their abuse become available.

Pharmacological strategies have developed as either agonist-antagonist or agonist-additional active ingredient combinations. Agonist-antagonist formulations can be considered pharmacodynamic strategies because they act to reduce reward at the receptor level. An example of such a strategy is Embeda (King Pharmaceuticals, Bristol, Tenn.), which combines morphine with an antagonist. If this formulation is ingested normally, the naltrexone remains latent; if it is crushed, the naltrexone is released and reduces the effects of the morphine.

Another added-ingredient approach is the use of the vitamin niacin, which is designed to induce unpleasant, but temporary, symptoms if too many tablets are consumed. The efficacy of agonist-antagonist and agonist-additional active ingredient formulations in reducing misuse and abuse of opioids in the real world is still untested. However, these approaches represent incremental advantages in the reformulation of opioid therapies.

Abuse-resistant mechanisms use physical strategies that make extracting the active drug from its formulation more difficult. One such formulation is a controlled-release oxycodone, Remoxy (King Pharmaceuticals) which sequesters oxycodone in a high-viscosity matrix. This investigational drug is intended to resist physical manipulation and chemical extraction used to alter drug delivery to unintended routes, such as injection, snorting, and other common methods of abuse. The evidence suggests that this gel cap formulation provides a stable 12-hour dose of oxycodone and is difficult to extract by chewing, crushing, freezing, and crushing or dissolving in water, alcohol, or other common beverages. These extractability studies establish an inherent difficulty in defeating the controlled-release mechanism of this formulation.

Opioids with a reduced abuse potential are one of the most important unmet needs in the management of chronic pain. Whether through pharmacodynamic or physical means, abuse-deterrent and abuse-resistant formulations may help to meet that need, although their actual impact will not be known until postmarketing data can be collected. This could restore the confidence of physicians in prescribing long-acting opioids and present an opportunity to increase access to opioid medications in minority and low-income communities, where such access has been limited. Of course, access to these medications also will depend on their cost and the willingness of health care and prescription organizations to include them on formularies. As part of a comprehensive risk management plan, these formulations may help physicians to better balance optimal analgesia with reduced risk of prescription misuse and abuse.

Metopon was an early opioid derivative with promising properties distinguishing it from other of the commercial opioids including analgesic effectiveness at least double to that of morphine and its duration of action was about equal to that of morphine. Furthermore, metopon was nearly devoid of emetic action; tolerance to it appeared to develop more slowly and to disappear more quickly, and physical dependence built up more slowly than with morphine; therapeutic analgesic doses produced little or no respiratory depression and much less mental dullness than morphine. Unfortunately, metopon suffered from low bioavailability and reduced potency relative to morphine. See Eddy, N., Metopon Hydrochloride An Experiment in Public Health, Public Health Reports, 64(4) 1949, p 93-103. The prodrugs of the present invention possess dramatically enhanced bioavailability while maintaining the reduced respiratory depression, emesis and tolerance observed with metopon.

### SUMMARY OF THE INVENTION

The invention relates to a compound of the Formula

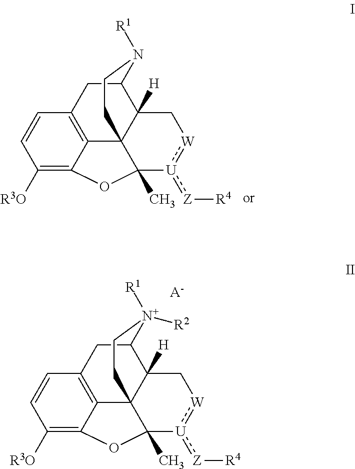

R¹ is alkyl, preferably methyl, ethyl, isopropyl or cyclopropyl;

R² is O⁻, CHR⁵OPO₃X₂, CHR⁵OPO₃H₂, CHR⁵OPO(OCHR⁵OCO₂R⁶)₂, CHR⁵OPO(OCHR⁵OCOR⁶)₂, CHR⁵OCO₂R⁶ (wherein R⁶ is other than H), or CHR⁵OCOR⁶;

R³ is H (except that R¹ can not be CH₃ in Formula I when U—Z is carbonyl); alkyl preferably CH₃ (except that R¹ can not be CH₃ in Formula I when U—Z is carbonyl), or R⁷;

R⁴ is absent when U—Z is carbonyl, or

Is COR⁷ when U—W is a C—C double bond and U—Z is a single C—O bond, or

Is H, alkyl, OH or O-alkyl when Z is N and U—Z is carbon-nitrogen double bond and U—W is a single bond;

R⁵ wherever it occurs is independently H or alkyl (preferably methyl, ethyl, isopropyl or cyclopropyl);

R⁶ wherever it occurs is independently H, alkyl, cycloalkyl, phenylalkyl, heteroalkyl, heterocycloalkyl, aryl, heteroaryl, or a sugar such as galactose;

R⁷ wherever it occurs is independently selected from alkyl (with the proviso that alkyl can not be CH₃ in formula I when U—Z is carbonyl), cycloalkyl, phenylalkyl, heteroalkyl, heterocycloalkyl, CHR⁵OCO₂R⁶, CHR⁵OCOR⁶, CHR⁵₂, (CH₂)ₙCO₂X, (CH₂)ₙCO₂H, (CH₂)ₙNR⁶₂, CHR⁵OPO₃H, CHR⁵OPO₃X, CHR⁶NHR⁶, -alkylCO₂X, alkylCO₂H, CHR⁵OCOR⁶, CHR⁵OCO₂R⁶ (wherein R⁶ is other than H), CHR⁵OPO(OCHR⁵OCOR⁶)₂, CHR⁵OCOCHR⁵NH₂, COR⁸, COCHCHCO₂H, COCHCHCO₂X, COCH₂CH₂SO₃X,

CO(CH₂)ₙCONHCO(CH₂)ₙCONHCH₂CH₂SO₃X, CONCH₃CH₂CF₂CH₂NH₂, COCHR⁵NR⁶R⁶, CO₂R⁶, COR⁶, CONR⁶₂, COCOR⁶, COCOOR⁶, COSR⁶, CSR⁶, CSOR⁶, SO₂NR⁵R⁶, SO₂OR⁶, SO₂R⁶, CONHCHR⁵CO₂CH₂CHNH₂CO₂H, CONHCHR⁵CO₂CH₂CHNH₂CO₂X, CONHCHR⁵CO₂CH₂CHNH₂CO₂H, CONHCHR⁵CO₂CHNH₂CO₂X, CONHCHR⁵CO₂CH₂CHNH₂CO₂H, CHR⁵OPO(OCHR⁵OCO₂R⁶)₂, CHR⁵OPO(OCHR⁵OCOR⁶)₂, CON(CH₃)(CH₂)₂NHCOCH(CH₃)CH2CH2CH2(NCOCH₃)CNHNH₂, CO(CH₂)₃CONHCHR⁵CO₂H, PO₃X₂, PO₃H₂, PO₃R⁶₂, COCHR⁹(NCOCHR⁹)ₙNH₂,

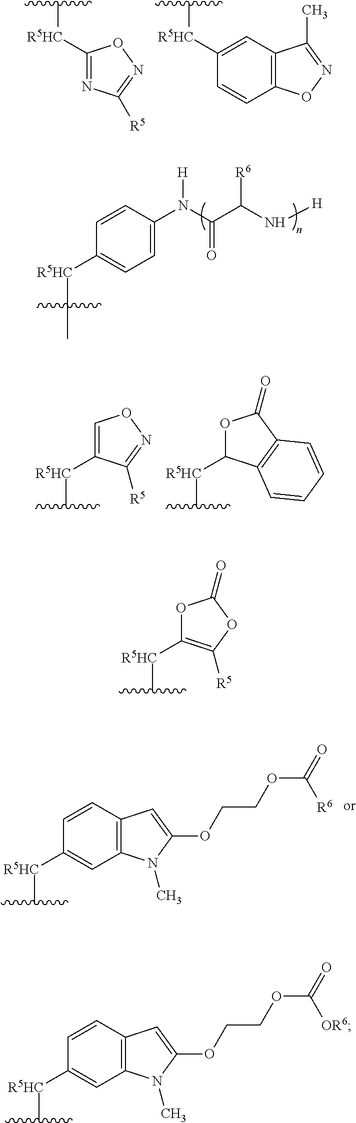

R⁸ is alkyl, cycloalkyl, phenylalkyl, heteroalkyl, heterocycloalkyl, aryl, heteroaryl,

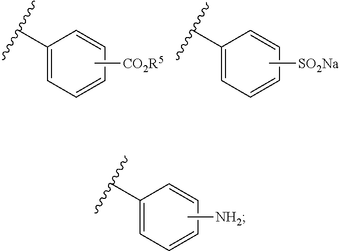

R⁹ is a 1 to 6, more preferably 1 to 4, natural, synthetic, racemic, L-, or D-amino acid group;

A is an anion selected from the group consisting of: Br—, Cl—, I—, R7CO2-(lactate, acetate, tartrate, valerate), H₂PO₄⁻, NO₃⁻, and R⁶SO₃⁻

X is a cation independently selected from the group consisting of:

Li⁺, Na⁺, K⁺, Mg⁺, ⁺NH₃R⁷, ⁺NH₂R⁷₂, ⁺NHR⁷₃, ⁺NR⁷₄, and ⁺NH₃(CH₂)ₙOH;

U is C;

W is CH₂ or CH;

Z═O; NH; NOH; NCH₃; NOR⁵;

Each n is an integer independently selected from 1 to 6.

An embodiment of the invention relates to a compound of Formula I

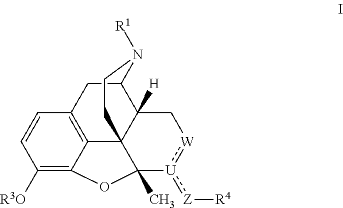

R¹ is methyl, ethyl, isopropyl or cyclopropyl;

R² is O⁻, CHR⁵OPO₃X₂, CHR⁵OPO₃H₂, CHR⁵OPO(OCHR⁵OCO₂R⁶)₂, CHR⁵OPO(OCHR⁵OCOR⁶)₂, CHR⁵OCO₂R⁶ (wherein R⁶ is other than H), or CHR⁵OCOR⁶;

R³ is H (except that R¹ can not be CH₃ in Formula I when U—Z is carbonyl); alkyl preferably CH₃ (except that R¹ can not be CH₃ in Formula I when U—Z is carbonyl), or R⁷;

R⁴ is absent when U—Z is carbonyl, or

Is COR⁷ when U—W is C—C double bond and U—Z is single C—O bond, or

Is H, alkyl, OH or O-alkyl when Z is N and U—Z is carbon-nitrogen double bond and U—W is a single bond;

R⁵ is H or alkyl (preferably methyl, ethyl, isopropyl or cyclopropyl);

R⁶ is H, alkyl, cycloalkyl, phenylalkyl, heteroalkyl, heterocycloalkyl, aryl, heteroaryl, or a sugar such as galactose;

R⁷ wherever it occurs is independently selected from alkyl (with the proviso that alkyl can not be CH₃ in formula I when U—Z is carbonyl), cycloalkyl, phenylalkyl, heteroalkyl, heterocycloalkyl, CHR⁵OCO₂R⁶, CHR⁵OCOR⁶, CHR⁵₂, (CH₂)ₙCO₂X, (CH₂)ₙCO₂H, (CH₂)ₙNR⁶₂, CHR⁵OPO₃H, CHR⁵OPO₃X, CHR⁶NHR⁶, alkylCO₂X, alkylCO₂H, CHR⁵OCOR⁶, CHR⁵OCO₂R⁶ (wherein R⁶ is other than H), CHR⁵OPO(OCHR⁵OCOR⁶)₂, CHR⁵OCOCHR⁵NH₂, COR⁸, COCHCHCO₂H, COCHCHCO₂X, COCH₂CH₂SO₃X,

CO(CH₂)ₙCONHCO(CH₂)ₙCONHCH₂CH₂SO₃X, CONMeCH₂CF₂CH₂NH₂, COCHR⁵NR⁶R⁶, CO₂R⁶, COR⁶, CONR⁶₂, COCOR⁶, COCOOR⁶, COSR⁶, CSR⁶, CSOR⁶, SO₂NR⁵R⁶, SO₂OR⁶, SO₂R⁶, CONHCHR⁵CO₂CH₂CHNH₂CO₂H, CONHCHR⁵CO₂CH₂CHNH₂CO₂X, CONHCHR⁵CO₂CH₂CHNH₂CO₂H, CHR⁵OPO(OCHR⁵OCO₂R⁶)₂, CHR⁵OPO(OCHR⁵OCOR⁶)₂, CON(CH₃)(CH₂)₂NHCOCH(CH₃)CH2CH2CH2(NCOCH₃)CNHNH₂, CO(CH₂)₃CONHCHR⁵CO₂H, PO₃X₂, PO₃H₂, PO₃R⁶₂, COCHR⁹(NCOCHR⁹)ₙNH₂,

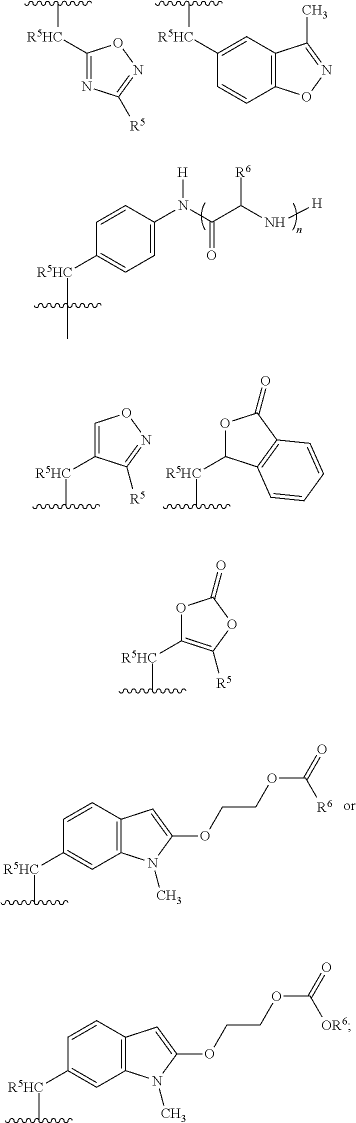

R⁸ is alkyl, cycloalkyl, phenylalkyl, heteroalkyl, heterocycloalkyl, aryl, heteroaryl,

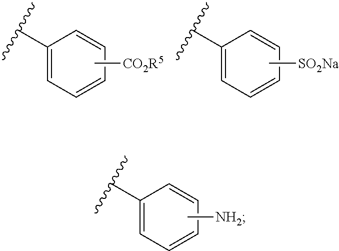

R⁹ is a 1 to 6, more preferably 1 to 4, natural, synthetic, racemic, L-, or D-amino acid group;

A is an anion selected from the group consisting of: Br⁻, Cl⁻, I⁻, R⁷CO₂⁻ (lactate, acetate, tartrate, valerate), H₂PO₄⁻, NO₃⁻, and R⁶SO₃⁻

X is a cation independently selected from the group consisting of: Li⁺, Na⁺, K⁺, Mg⁺, ⁺NH₃R⁷, ⁺NH₂R⁷₂, ⁺NHR⁷₃, ⁺NR⁷₄, and ⁺NH₃(CH₂)ₙOH;

U is C;

W is CH₂ or CH;

Z═O; NH; NOH; NMe; NOR⁵;

Each n is an integer independently selected from 1 to 6.

An embodiment of the invention relates to a compound of Formula I wherein R³ is H, U—Z is carbonyl and R¹ is ethyl, isopropyl or cyclopropyl.

Another embodiment of the invention relates to a compound of Formula I wherein R³ is alkyl, preferably CH₃, U—Z is carbonyl and R¹ is ethyl, isopropyl or cyclopropyl.

Another embodiment of the invention relates to a compound of Formula I wherein R³ is H, U—W is a C—C double bond, U—Z is a single C—O bond, R⁴ is COR⁷, and R¹ is methyl, ethyl, isopropyl or cyclopropyl; and

R⁷ is selected from alkyl, cycloalkyl, phenylalkyl, heteroalkyl, heterocycloalkyl, CHR⁵OCO₂R⁶, CHR⁵OCOR⁶, CHR⁵₂, (CH₂)ₙCO₂X, (CH₂)ₙCO₂H, (CH₂)ₙNR⁶₂, CHR⁵OPO₃H, CHR⁵OPO₃X, CHR⁶NHR⁶, alkylCO₂X, alkylCO₂H, CHR⁵OCOR⁶, CHR⁵OCO₂R⁶ (wherein R⁶ is other than H), CHR⁵OPO(OCHR⁵OCOR⁶)₂, CHR⁵OCOCHR⁵NH₂, COR⁸, COCHCHCO₂H, COCHCHCO₂X, COCH₂CH₂SO₃X,

CO(CH₂)ₙCONHCO(CH₂)ₙCONHCH₂CH₂SO₃X, CONMeCH₂CF₂CH₂NH₂, COCHR⁵NR⁶R⁶, CO₂R⁶, COR⁶, CONR⁶₂, COCOR⁶, COCOOR⁶, COSR⁶, CSR⁶, CSOR⁶, SO₂NR⁵R⁶, SO₂OR⁶, SO₂R⁶, CONHCHR⁵CO₂CH₂CHNH₂CO₂H, CONHCHR⁵CO₂CH₂CHNH₂CO₂X, CONHCHR⁵CO₂CH₂CHNH₂CO₂H, CHR⁵OPO(OCHR⁵OCO₂R⁶)₂, CHR⁵OPO(OCHR⁵OCOR⁶)₂, CON(CH₃)(CH₂)₂NHCOCH(CH₃)CH2CH2CH2(NCOCH₃)CNHNH₂, CO(CH₂)₃CONHCHR⁵CO₂H, PO₃X₂, PO₃H₂, PO₃R⁶₂, COCHR⁹(NCOCHR⁹)ₙNH₂,

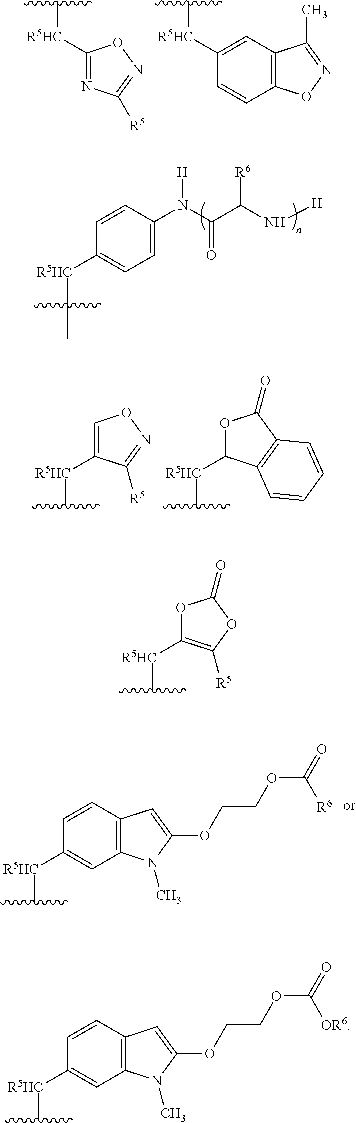

Another embodiment of the invention relates to a compound of Formula I wherein R³ is R⁷.

Another embodiment of the invention relates to a compound of Formula I wherein R³ is R⁷; U—Z is carbonyl and R¹ is ethyl, isopropyl or cyclopropyl.

An embodiment of the invention relates to a compound of Formula I wherein R³ is R⁷; U—W is a C—C double bond, U—Z is a single C—O bond, R⁴ is COR⁷.

Another embodiment of the invention relates to a compound of Formula II

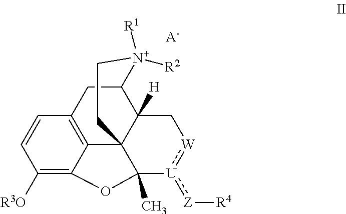

R¹ is alkyl, preferably methyl, ethyl, isopropyl or cyclopropyl;

R² is O⁻, CHR⁵OPO₃X₂, CHR⁵OPO₃H₂, CHR⁵OPO(OCHR⁵OCO₂R⁶)₂, CHR⁵OPO(OCHR⁵OCOR⁶)₂, CHR⁵OCO₂R⁶ (wherein R⁶ is other than H), or CHR⁵OCOR⁶;

R³ is H, alkyl (preferably CH₃), or R⁷;

R⁴ is absent when U—Z is carbonyl, or

Is COR⁷ when U—W═C—C double bond and U—Z=single C—O bond, or

Is H, alkyl, OH or O-alkyl when Z is N and U—Z is carbon-nitrogen double bond and U—W is a single bond;

R⁵ is H or alkyl (preferably methyl, ethyl, isopropyl or cyclopropyl);

R⁶ is H, alkyl, cycloalkyl, phenylalkyl, heteroalkyl, heterocycloalkyl, aryl, heteroaryl, or a sugar such as galactose;

R⁷ wherever it occurs is independently selected from alkyl, cycloalkyl, phenylalkyl, heteroalkyl, heterocycloalkyl, CHR⁵OCO₂R⁶, CHR⁵OCOR⁶, CHR⁵₂, (CH₂)ₙCO₂X, (CH₂)ₙCO₂H, (CH₂)ₙNR⁶₂, CHR⁵OPO₃H, CHR⁵OPO₃X, CHR⁶NHR⁶, alkylCO₂X, alkylCO₂H, CHR⁵OCOR⁶, CHR⁵OCO₂R⁶ (wherein R⁶ is other than H), CHR⁵OPO(OCHR⁵OCOR⁶)₂, CHR⁵OCOCHR⁵NH₂, COR⁸, COCHCHCO₂H, COCHCHCO₂X, COCH₂CH₂SO₃X, CO(CH₂)ₙCONHCO(CH₂)ₙCONHCH₂CH₂SO₃X, CONMeCH₂CF₂CH₂NH₂, COCHR⁵NR⁶R⁶, CO₂R⁶, COR⁶, CONR⁶₂, COCOR⁶, COCOOR⁶, COSR⁶, CSR⁶, CSOR⁶, SO₂NR⁵R⁶, SO₂OR⁶, SO₂R⁶, CONHCHR⁵CO₂CH₂CHNH₂CO₂H, CONHCHR⁵CO₂CH₂CHNH₂CO₂X, CONHCHR⁵CO₂CH₂CHNH₂CO₂H, CHR⁵OPO(OCHR⁵OCO₂R⁶)₂, CHR⁵OPO(OCHR⁵OCOR⁶)₂, CON(CH₃)(CH₂)₂NHCOCH(CH₃)CH2CH2CH2(NCOCH₃)CNHNH₂, CO(CH₂)₃CONHCHR⁵CO₂H, PO₃X₂, PO₃H₂, PO₃R⁶₂, COCHR⁹(NCOCHR⁹)ₙNH₂,

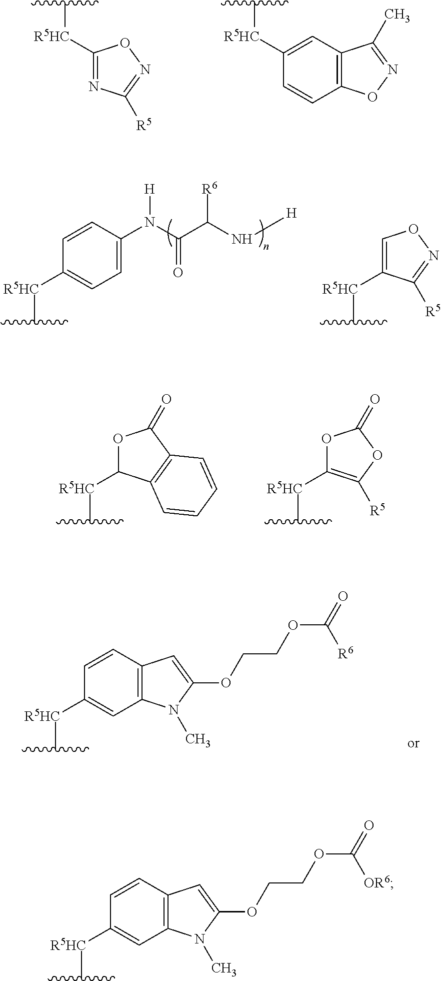

R⁸ is alkyl, cycloalkyl, phenylalkyl, heteroalkyl, heterocycloalkyl, aryl, heteroaryl,

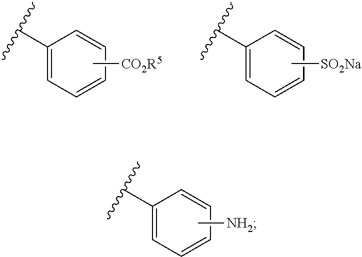

R⁹ is a 1 to 6, more preferably 1 to 4, natural, synthetic, racemic, L-, or D-amino acid group;

A is an anion selected from the group consisting of: Br⁻, Cl⁻, I⁻, R⁷CO₂⁻ (lactate, acetate, tartrate, valerate), H₂PO₄⁻, NO₃⁻, and R⁶SO₃⁻

X is a cation independently selected from the group consisting of: Li⁺, Na⁺, K⁺, Mg⁺, ⁺NH₃R⁷, ⁺NH₂R⁷₂, ⁺NHR⁷₃, ⁺NR⁷₄, and ⁺NH₃(CH₂)ₙOH;

U is C;

W is CH₂ or CH;

Z═O; NH; NOH; NMe; NOR⁵;

Each n is an integer independently selected from 1 to 6.

Another embodiment of the invention relates to a compound of Formula II wherein R⁴ is absent and U—Z is carbonyl.

Another embodiment of the invention relates to a compound of Formula II wherein R³ is H or alkyl.

Another embodiment of the invention relates to a compound of Formula II wherein R³ is R⁷.

Another embodiment of the invention relates to a compound of Formula II wherein U—W is a C—C double bond; U—Z is a single C—O bond and R⁴ is COR⁷.

Another embodiment of the invention relates to a compound of Formula II wherein R³ is H or alkyl.

Another embodiment of the invention relates to a compound of Formula II wherein Z is N, U—Z is a carbon-nitrogen double bond, U—W is a C—C single bond; and R⁴ is H, alkyl, OH or O-alkyl.

Another embodiment of the invention relates to a compound of Formula II wherein R³ is H or alkyl.

Another embodiment of the invention relates to a composition of any of the aforesaid embodiments of compounds of Formula I or II wherein said composition is in tablet, capsule, oral solution, or oral suspension dosage form.

Another embodiment of the invention relates to a method of treating acute or chronic pain comprising administering to a patient the aforesaid composition.

Another embodiment of the invention relates to a method of treating acute or chronic pain comprising administering to a patient a compound of Formula I or II.

Another embodiment of the invention relates to a composition of a compound of Formula I or II formulated into a tablet, a capsule, an oral solution, or an oral suspension.

Another embodiment of the invention relates to a composition of a compound of Formula I or II wherein said composition is in tablet or capsule dosage form.

Alkyl means unsubstituted and substituted, straight-chain and branched-chain alkyls and cyclic having from 1 to 20 carbon atoms; straight-chain alkyl is optionally substituted with 1 or 2 substituents independently selected from the group consisting of halo, hydroxy, alkoxy(alkoxy)x, hydroxyalkoxy(alkoxy)x, amino, monoalkylamino, dialkylamino, nitro, carboxyl, alkoxycarbonyl, and cyano, wherein x is an integer from 0 to 3 and the alkoxy contains from 1 to 5 carbon atoms.

Cycloalkyl means cyclopropyl, cyclobutyl, cyclopentyl and cyclohexyl; and said cyclic alkyl is optionally substituted with 1 or 2 substituents independently selected from the group consisting of halo, hydroxy, alkoxy(alkoxy)x, hydroxyalkoxy(alkoxy)x, amino, monoalkylamino, dialkylamino, nitro, carboxyl, alkoxycarbonyl, and cyano, wherein x is an integer from 0 to 3 and the alkoxy portion of the alkoxycarbonyl contains from 1 to 5 carbon atoms.

Phenylalkyl is selected from the group consisting of benzyl, phenylethyl and phenylpropyl; and the phenyl portion of the phenylalkyl is optionally substituted with 1 to 3 substituents independently selected from the group consisting of alkyl, hydroxy, alkoxy, halo, amino, monoalkylamino, dialkylamino, nitro, carboxyl, alkoxycarbonyl and cyano, wherein the phenyl portion of the phenylalkyl is unsubstituted or substituted;

Heteroalkyl means a straight or branched-chain having from one to 20 carbon atoms and one or more heteroatoms selected from nitrogen, oxygen, or sulphur, wherein the nitrogen and sulphur atoms may optionally be oxidized, i.e., in the form of an N-oxide or an S-oxide.

Heterocycloalkyl means a monocyclic or multicyclic ring system (which may be saturated or partially unsaturated), including fused and spiro rings, of about five to about 10 elements wherein one or more of the elements in the ring system is an element other than carbon and is selected from nitrogen, oxygen, silicon, or sulphur atoms.

Aryl means phenyl or napthyl wherein the phenyl or napthyl moiety is optionally substituted with 1 to 3 substituents independently selected from the group consisting of alkyl, hydroxy, alkoxy, halo, amino, monoalkylamino, dialkylamino, nitro, carboxyl, alkoxycarbonyl and cyano groups.

Heteroaryl means a five to about a 14-membered aromatic monocyclic or multicyclic hydrocarbon ring system, including fused and spiro rings, in which one or more of the elements in the ring system is an element other than carbon and is selected from nitrogen, oxygen, silicon, or sulphur and wherein an N atom may be in the form of an N-oxide.

Natural, synthetic, racemic, L-, or D-amino acid group as used herein refers to a substituent containing 1 to 20 amino acid. When two or more amino acids are linked together the group is known as a polypeptide. The polypeptide may be (i) an oligopeptide, (ii) a homopolymer of one of the twenty naturally occurring amino acids, (iii) a heteropolymer of two or more naturally occurring amino acids, (iv) a homopolymer of a synthetic amino acid, (v) a heteropolymer of two or more synthetic amino acids or (vi) a heteropolymer of one or more naturally occurring amino acids and one or more synthetic amino acids. Polypeptides include the twenty naturally occurring amino acids. Specific polypeptides include one or more amino acids selected from glutamic acid, aspartic acid, arginine, asparagine, cysteine, lysine, threonine, or serine. Such peptides may be attached through the C-terminus or the N-terminus.

Selection of the particular amino acids will depend on the physical properties desired. For instance, properties such as bulk, lipophilicity, and hydrophilicity may be optimized according to selection parameters known to those skilled in the art.

Sugar or saccharide as used herein refers to a monosaccharide, a disaccharide, polysaccharide or sugar alcohol. Saccharide includes galactose, fructose, glucose, maltose, cellobiose, gentiobiose, melibiose, lactose, turanose, sophorose, trehalose, isotrehalose, isosaccharose, white sugar, mannitol, sorbitol, xylitol or inositol.

Another embodiment of the invention relates to an oral pharmaceutical preparation containing a therapeutically effective amount of a compound of formula I or II or a salt thereof for once daily administration.

Another embodiment of the invention relates to a composition containing particles which have a core containing a compound of Formula I or II or a salt thereof coated with a barrier layer. The barrier layer is formed from a coating liquid that contains a least one water insoluble barrier forming component selected from a group consisting of ethyl cellulose, copolymers of acrylic and methacrylic esters and natural or synthetic waxes, and a plasticizer.

The compounds of Formula I and II may exist in the form of pharmaceutically acceptable salts such as, e.g., acid addition salts and base addition salts of the compounds of Formula I and II. The phrase “pharmaceutically acceptable salt(s)”, as used herein, unless otherwise indicated, includes salts of acidic or basic groups which may be present in the compounds of Formula I or II.

Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002).

As used herein the terms ““Formula I or II” and “Formula I or II or pharmaceutically acceptable salts thereof” are defined to include all forms of the compounds of Formula I or II, including hydrates, solvates, isomers, crystalline and non-crystalline forms, isomorphs, polymorphs, and metabolites thereof.

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

The compounds of the invention may also exist in unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallisation, by recrystallisation from solvents, or by physically grinding the components together—see Chem Commun, 17, 1889-1896, by O. Almarsson and M. J. Zaworotko (2004). For a general review of multi-component complexes, see J Pharm Sci, 64 (8), 1269-1288, by Haleblian (August 1975).

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

Hereinafter all references to compounds of formula I or II include references to salts, solvates, multi-component complexes and liquid crystals thereof and to solvates, multi-component complexes and liquid crystals of salts thereof.

The compounds of the invention include compounds of formula I or II as hereinbefore defined, including all polymorphs and crystal habits thereof, and isomers thereof (including optical, geometric and tautomeric isomers) as hereinafter defined and isotopically-labeled compounds of formula I or II.

The compounds of Formula I or II may have asymmetric carbon atoms and may exist as two or more stereoisomers. The carbon-carbon bonds of the compounds of Formula I and II may be depicted herein using a solid line ( [symbol] ), a solid wedge ( [symbol] ) or a dotted wedge ( [symbol] ). The use of a solid line to depict bonds to asymmetric carbon atoms is meant to indicate that all possible stereoisomers (e.g. specific enantiomers, racemic mixtures, etc.) at that carbon atom are included. The use of either a solid or dotted wedge to depict bonds to asymmetric carbon atoms is meant to indicate that only the stereoisomer shown is meant to be included. It is possible that compounds of Formula I or II may contain more than one asymmetric carbon atom. In those compounds, the use of a solid line to depict bonds to asymmetric carbon atoms is meant to indicate that all possible stereoisomers are meant to be included. For example, unless stated otherwise, it is intended that the compounds of Formula I or II can exist as enantiomers and diastereomers or as racemates and mixtures thereof. The use of a solid line to depict bonds to one or more asymmetric carbon atoms in a compound of Formula I and II and the use of a solid or dotted wedge to depict bonds to other asymmetric carbon atoms in the same compound is meant to indicate that a mixture of diastereomers is present.

Stereoisomers of Formula I or II include cis and trans isomers, optical isomers such as R and S enantiomers, diastereomers, geometric isomers, rotational isomers, conformational isomers, and tautomers of the compounds of Formula I or II, including compounds exhibiting more than one type of isomerism; and mixtures thereof (such as racemates and diastereomeric pairs). Also included are acid addition or base addition salts wherein the counterion is optically active, for example, d-lactate or l-lysine, or racemic, for example, dl-tartrate or dl-arginine.

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

The compounds of the Formula I or II may exhibit the phenomena of tautomerism and structural isomerism. For example, the compounds of Formula I may exist in several tautomeric forms, including the enol and imine form, and the keto and enamine form and geometric isomers and mixtures thereof. All such tautomeric forms are included within the scope of compounds of Formula I or II. Tautomers exist as mixtures of a tautomeric set in solution. In solid form, usually one tautomer predominates. Even though one tautomer may be described, the present invention includes all tautomers of the compounds of Formula I or II.

The present invention includes all pharmaceutically acceptable isotopically-labelled compounds of formula I or II wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of formula I or II, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula I or II can generally be prepared by conventional techniques known to those skilled in the art or by processes analogous to those described in the accompanying Examples and Preparations using an appropriate isotopically-labeled reagent in place of the non-labeled reagent previously employed.

Other embodiments of the present invention relate to the following compounds of Formula I or II:

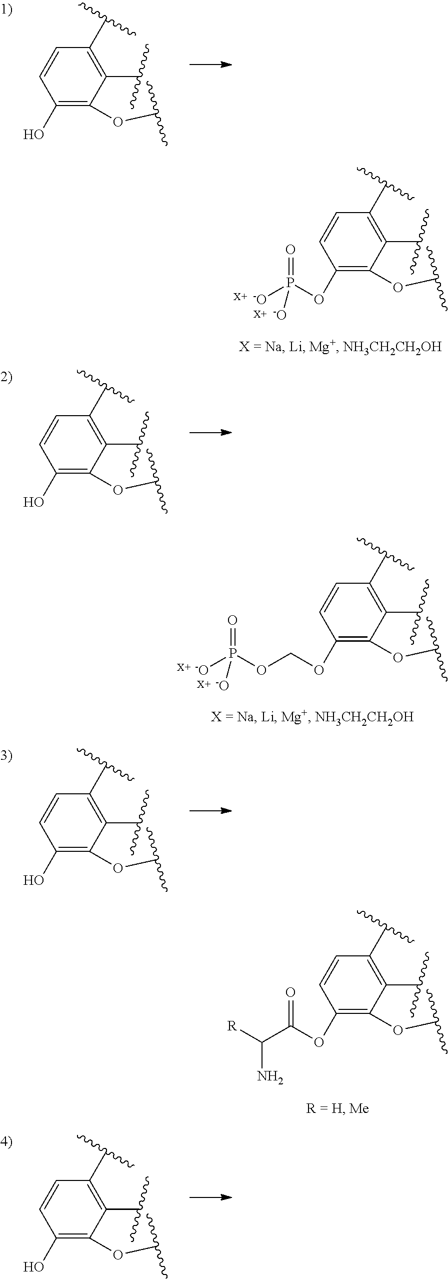

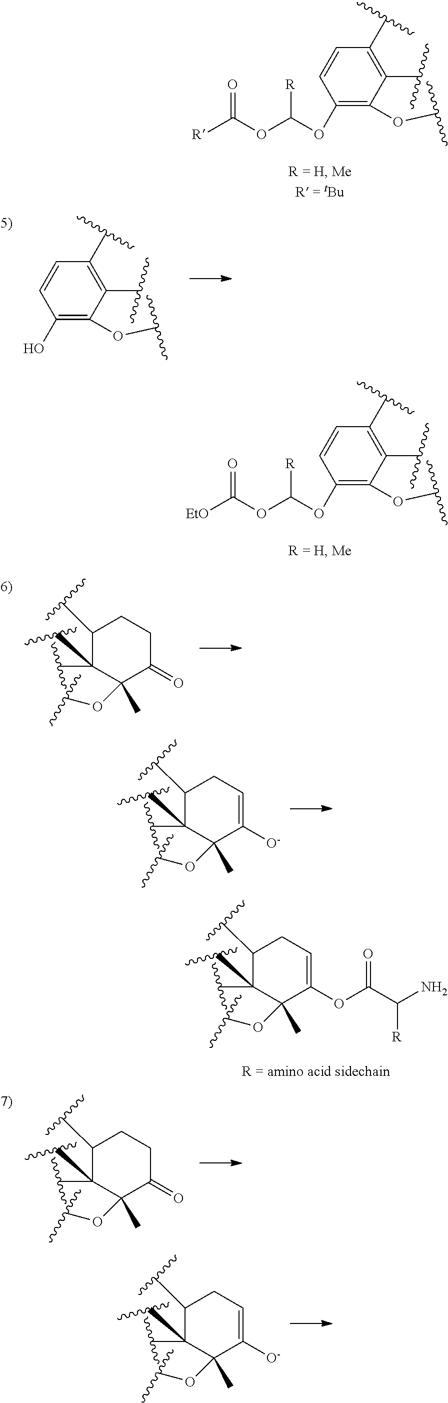

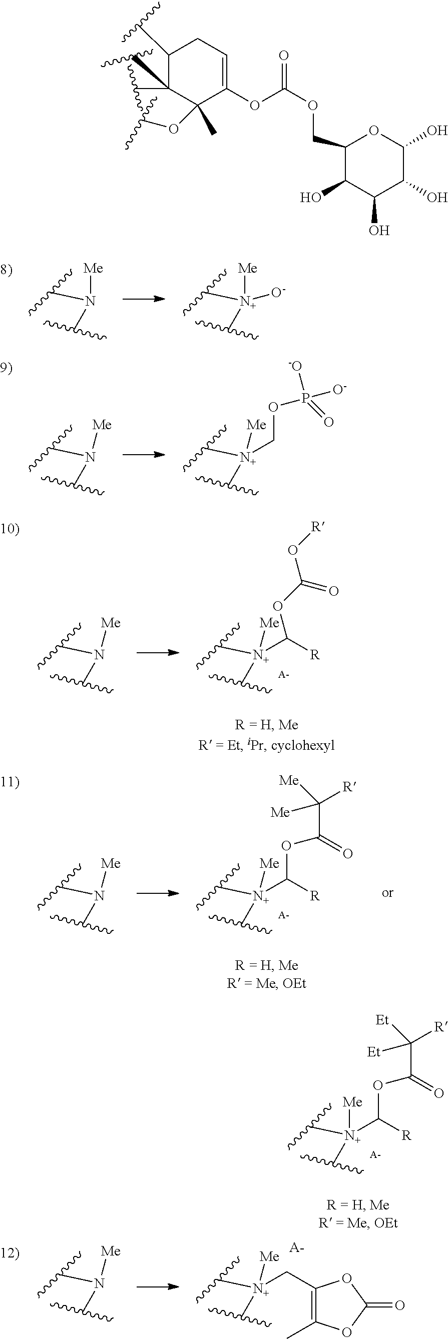

Specific compounds of the invention of interest include:

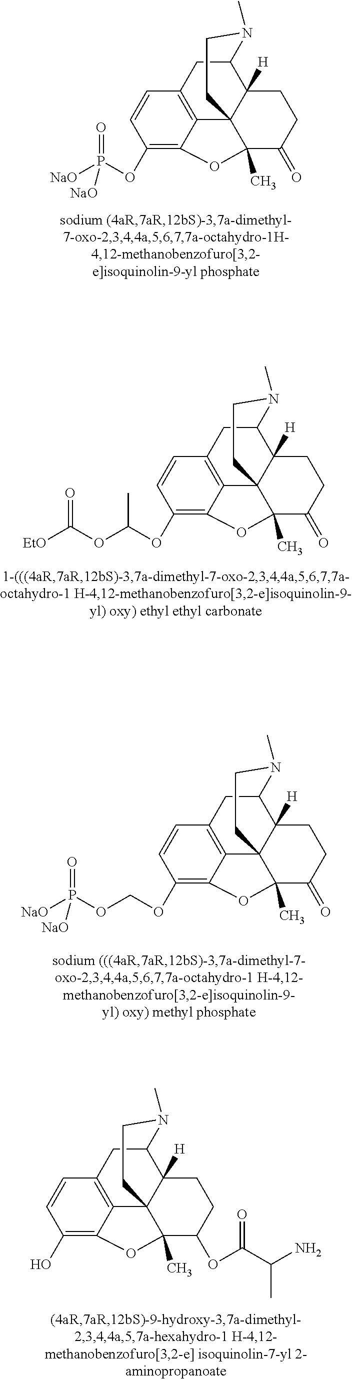

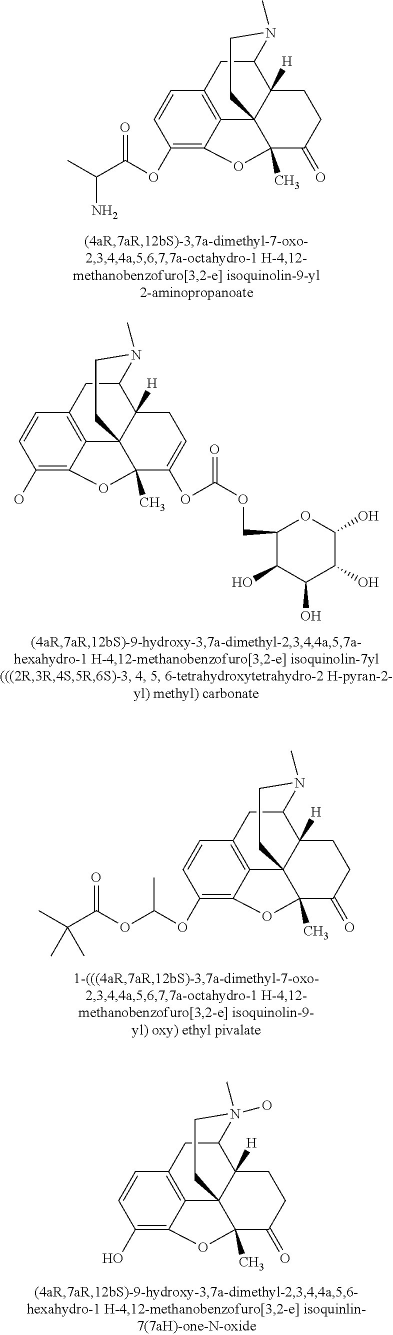

Yet other compounds of interest include:

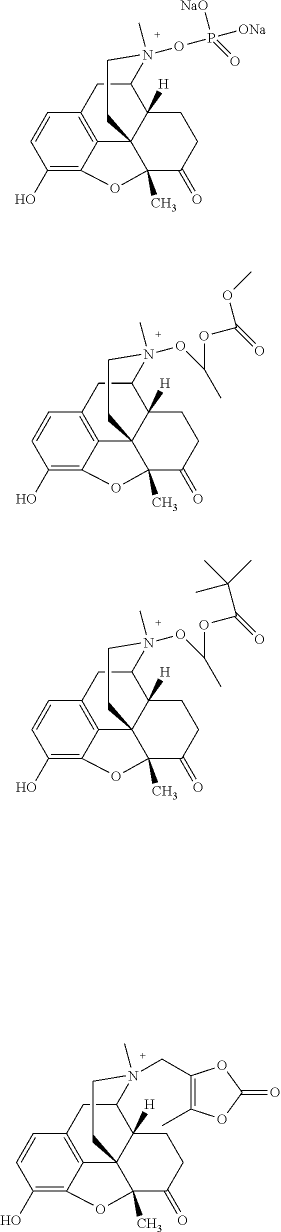

The invention also relates to compositions comprising a compound of Formula I or II or an acceptable salt thereof (e.g., pharmaceutical compositions). Accordingly, in one embodiment, the invention relates to a pharmaceutical composition comprising a compound of Formula I or II, a pharmaceutically acceptable carrier and, optionally, at least one additional medicinal or pharmaceutical agent.

In yet further embodiments, the pharmaceutical composition of the present invention in addition to the compound of Formula I or II may further include a non-opioid drug. Such non-opioid drugs would preferably provide additional analgesia, and include, for example, aspirin; acetaminophen; non-steroidal anti-inflammatory drugs (“NSAIDS”), e.g., ibuprofen, ketoprofen, etc.; N-methyl-D-aspartate (NMDA) receptor antagonists, e.g., a morphinan such as dextromethorphan or dextrorphan, or ketamine; cyclooxygenase-II inhibitors (“COX-II inhibitors”); and/or glycine receptor antagonists.

Suitable non-steroidal anti-inflammatory agents, include ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam, isoxicam, pharmaceutically acceptable salts thereof, mixtures thereof, and the like. Useful dosages of these drugs are well known to those skilled in the art.

N-methyl-D-aspartate (NMDA) receptor antagonists are well known in the art, and encompass, for example, morphinans such as dextromethorphan or dextrorphan, ketamine, or pharmaceutically acceptable salts thereof. For purposes of the present invention, the term “NMDA antagonist” is also deemed to encompass drugs that at least partially inhibit a major intracellular consequence of NMDA-receptor activation, e.g. a ganglioside such as GM.sub.1 or GT.sub.1b, a phenothiazine such as trifluoperazine or a naphthalenesulfonamide such as N-(6-aminothexyl)-5-chloro-1-naphthalenesulfonamide. These drugs are stated to inhibit the development of tolerance to and/or dependence on addictive drugs, e.g., narcotic analgesics such as morphine, codeine, etc. in U.S. Pat. Nos. 5,321,012 and 5,556,838 (both to Mayer, et al.), and to treat chronic pain in U.S. Pat. No. 5,502,058 (Mayer, et al.). The NMDA antagonist may be included alone, or in combination with a local anesthetic such as lidocaine, as described in these Mayer, et al. patents.

The treatment of chronic pain via the use of glycine receptor antagonists and the identification of such drugs is described in U.S. Pat. No. 5,514,680 (Weber, et al.).

COX-2 inhibitors have been reported in the art and many chemical structures are known to produce inhibition of cyclooxygenase-2. COX-2 inhibitors are described, for example, in U.S. Pat. Nos. 5,616,601; 5,604,260; 5,593,994; 5,550,142; 5,536,752; 5,521,213; 5,475,995; 5,639,780; 5,604,253; 5,552,422; 5,510,368; 5,436,265; 5,409,944; and 5,130,311. Certain preferred COX-2 inhibitors include celecoxib, flosulide, meloxicam, 6-methoxy-2 naphthylacetic acid (6-MNA), nabumetone (prodrug for 6-MNA), nimesulide, or combinations thereof. Dosage levels of COX-2 inhibitor on the order of from about 0.005 mg to about 140 mg per kilogram of body weight per day are therapeutically effective in combination with the compounds of Formula I. Alternatively, about 0.25 mg to about 7 g per patient per day of a COX-2 inhibitor is administered in a combination.

In yet further embodiments, a non-opioid drug can be included which provides a desired effect other than analgesia, e.g., antitussive, expectorant, decongestant, antihistamine drugs, local anesthetics, and the like.

The pharmaceutical acceptable carrier may comprise any conventional pharmaceutical carrier or excipient. Suitable pharmaceutical carriers include inert diluents or fillers, water and various organic solvents (such as hydrates and solvates). The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Non-limiting examples of materials, therefore, include lactose or milk sugar and high molecular weight polyethylene glycols.

The pharmaceutical composition may, for example, be in a form suitable for oral administration as a tablet, capsule, pill, powder, sustained release formulations, solution suspension, for parenteral injection as a sterile solution, suspension or emulsion, for topical administration as an ointment or cream or for rectal administration as a suppository.

The pharmaceutical composition may be in unit dosage forms suitable for single administration of precise dosages.

In one embodiment the composition comprises a therapeutically effective amount of a compound of Formula I or II and a pharmaceutically acceptable carrier.

The compounds of Formula I or II are useful in the treatment of pain, particularly neuropathic, nociceptive and inflammatory pain.

Physiological pain is an important protective mechanism designed to warn of danger from potentially injurious stimuli from the external environment. The system operates through a specific set of primary sensory neurones and is activated by noxious stimuli via peripheral transducing mechanisms (see Millan, 1999, Prog. Neurobiol., 57, 1-164 for a review). These sensory fibres are known as nociceptors and are characteristically small diameter axons with slow conduction velocities. Nociceptors encode the intensity, duration and quality of noxious stimulus and by virtue of their topographically organised projection to the spinal cord, the location of the stimulus. The nociceptors are found on nociceptive nerve fibres of which there are two main types, A-delta fibres (myelinated) and C fibres (non-myelinated). The activity generated by nociceptor input is transferred, after complex processing in the dorsal horn, either directly, or via brain stem relay nuclei, to the ventrobasal thalamus and then on to the cortex, where the sensation of pain is generated.

Pain may generally be classified as acute or chronic. Acute pain begins suddenly and is short-lived (usually twelve weeks or less). It is usually associated with a specific cause such as a specific injury and is often sharp and severe. It is the kind of pain that can occur after specific injuries resulting from surgery, dental work, a strain or a sprain. Acute pain does not generally result in any persistent psychological response. In contrast, chronic pain is long-term pain, typically persisting for more than three months and leading to significant psychological and emotional problems. Common examples of chronic pain are neuropathic pain (e.g. painful diabetic neuropathy, postherpetic neuralgia), carpal tunnel syndrome, back pain, headache, cancer pain, arthritic pain and chronic post-surgical pain.

When a substantial injury occurs to body tissue, via disease or trauma, the characteristics of nociceptor activation are altered and there is sensitisation in the periphery, locally around the injury and centrally where the nociceptors terminate. These effects lead to a heightened sensation of pain. In acute pain these mechanisms can be useful, in promoting protective behaviors which may better enable repair processes to take place. The normal expectation would be that sensitivity returns to normal once the injury has healed. However, in many chronic pain states, the hypersensitivity far outlasts the healing process and is often due to nervous system injury. This injury often leads to abnormalities in sensory nerve fibers associated with maladaptation and aberrant activity (Woolf & Salter, 2000, Science, 288, 1765-1768).

Clinical pain is present when discomfort and abnormal sensitivity feature among the patient's symptoms. Patients tend to be quite heterogeneous and may present with various pain symptoms. Such symptoms include: 1) spontaneous pain which may be dull, burning, or stabbing; 2) exaggerated pain responses to noxious stimuli (hyperalgesia); and 3) pain produced by normally innocuous stimuli (allodynia—Meyer et al., 1994, Textbook of Pain, 13-44). Although patients suffering from various forms of acute and chronic pain may have similar symptoms, the underlying mechanisms may be different and may, therefore, require different treatment strategies. Pain can also therefore be divided into a number of different subtypes according to differing pathophysiology, including nociceptive, inflammatory and neuropathic pain.

Nociceptive pain is induced by tissue injury or by intense stimuli with the potential to cause injury. Pain afferents are activated by transduction of stimuli by nociceptors at the site of injury and activate neurons in the spinal cord at the level of their termination. This is then relayed up the spinal tracts to the brain where pain is perceived (Meyer et al., 1994, Textbook of Pain, 13-44). The activation of nociceptors activates two types of afferent nerve fibers. Myelinated A-delta fibers transmit rapidly and are responsible for sharp and stabbing pain sensations, whilst unmyelinated C fibers transmit at a slower rate and convey a dull or aching pain. Moderate to severe acute nociceptive pain is a prominent feature of pain from central nervous system trauma, strains/sprains, burns, myocardial infarction and acute pancreatitis, post-operative pain (pain following any type of surgical procedure), posttraumatic pain, renal colic, cancer pain and back pain. Cancer pain may be chronic pain such as tumour related pain (e.g. bone pain, headache, facial pain or visceral pain) or pain associated with cancer therapy (e.g. postchemotherapy syndrome, chronic postsurgical pain syndrome or post radiation syndrome). Cancer pain may also occur in response to chemotherapy, immunotherapy, hormonal therapy or radiotherapy. Back pain may be due to herniated or ruptured intervertabral discs or abnormalities of the lumber facet joints, sacroiliac joints, paraspinal muscles or the posterior longitudinal ligament. Back pain may resolve naturally but in some patients, where it lasts over 12 weeks, it becomes a chronic condition which can be particularly debilitating.

Neuropathic pain is currently defined as pain initiated or caused by a primary lesion or dysfunction in the nervous system. Nerve damage can be caused by trauma and disease and thus the term ‘neuropathic pain’ encompasses many disorders with diverse etiologies. These include, but are not limited to, peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy and vitamin deficiency. Neuropathic pain is pathological as it has no protective role. It is often present well after the original cause has dissipated, commonly lasting for years, significantly decreasing a patient's quality of life (Woolf and Mannion, 1999, Lancet, 353, 1959-1964). The symptoms of neuropathic pain are difficult to treat, as they are often heterogeneous even between patients with the same disease (Woolf & Decosterd, 1999, Pain Supp., 6, S141-S147; Woolf and Mannion, 1999, Lancet, 353, 1959-1964). They include spontaneous pain, which can be continuous, and paroxysmal or abnormal evoked pain, such as hyperalgesia (increased sensitivity to a noxious stimulus) and allodynia (sensitivity to a normally innocuous stimulus).

The inflammatory process is a complex series of biochemical and cellular events, activated in response to tissue injury or the presence of foreign substances, which results in swelling and pain (Levine and Taiwo, 1994, Textbook of Pain, 45-56). Arthritic pain is the most common inflammatory pain. Rheumatoid disease is one of the commonest chronic inflammatory conditions in developed countries and rheumatoid arthritis is a common cause of disability. The exact aetiology of rheumatoid arthritis is unknown, but current hypotheses suggest that both genetic and microbiological factors may be important (Grennan & Jayson, 1994, Textbook of Pain, 397-407). It has been estimated that almost 16 million Americans have symptomatic osteoarthritis (OA) or degenerative joint disease, most of whom are over 60 years of age, and this is expected to increase to 40 million as the age of the population increases, making this a public health problem of enormous magnitude (Houge & Mersfelder, 2002, Ann Pharmacother., 36, 679-686; McCarthy et al., 1994, Textbook of Pain, 387-395). Most patients with osteoarthritis seek medical attention because of the associated pain. Arthritis has a significant impact on psychosocial and physical function and is known to be the leading cause of disability in later life. Ankylosing spondylitis is also a rheumatic disease that causes arthritis of the spine and sacroiliac joints. It varies from intermittent episodes of back pain that occur throughout life to a severe chronic disease that attacks the spine, peripheral joints and other body organs.

Another type of inflammatory pain is visceral pain which includes pain associated with inflammatory bowel disease (IBD). Visceral pain is pain associated with the viscera, which encompass the organs of the abdominal cavity. These organs include the sex organs, spleen and part of the digestive system. Pain associated with the viscera can be divided into digestive visceral pain and non-digestive visceral pain. Commonly encountered gastrointestinal (GI) disorders that cause pain include functional bowel disorder (FBD) and inflammatory bowel disease (IBD). These GI disorders include a wide range of disease states that are currently only moderately controlled, including, in respect of FBD, gastro-esophageal reflux, dyspepsia, irritable bowel syndrome (IBS) and functional abdominal pain syndrome (FAPS), and, in respect of IBD, Crohn's disease, ileitis and ulcerative colitis, all of which regularly produce visceral pain. Other types of visceral pain include the pain associated with dysmenorrhea, cystitis and pancreatitis and pelvic pain.

It should be noted that some types of pain have multiple etiologies and thus can be classified in more than one area, e.g. back pain and cancer pain have both nociceptive and neuropathic components.

Other types of pain include: pain resulting from musculo-skeletal disorders, including myalgia, fibromyalgia, spondylitis, sero-negative (non-rheumatoid) arthropathies, non-articular rheumatism, dystrophinopathy, glycogenolysis, polymyositis and pyomyositis; heart and vascular pain, including pain caused by angina, myocardical infarction, mitral stenosis, pericarditis, Raynaud's phenomenon, scleredoma and skeletal muscle ischemia; head pain, such as migraine (including migraine with aura and migraine without aura), cluster headache, tension-type headache mixed headache and headache associated with vascular disorders; erythermalgia; and orofacial pain, including dental pain, otic pain, burning mouth syndrome and temporomandibular myofascial pain.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject.

Administration of the compounds of Formula I may be effected by any method that enables delivery of the compounds to the site of action. These methods include oral routes, intraduodenal routes, parenteral injection (including intravenous, subcutaneous, intramuscular, intravascular or infusion), topical, and rectal administration.

The present invention also relates to changing the pharmacokinetic and pharmacological properties of opioids, particularly metopon, through covalent modification. Covalent attachment of a chemical moiety to an opioid can change the rate and extent of absorption, metabolism, distribution, and elimination of the drug. When administered at a normal therapeutic dose the bioavailability (the time-versus-concentration curve; area under the curve; AUC) of the opioid is similar to that of the parent opioid compound. As the oral dose is increased, however, the bioavailability of the covalently modified opioid relative to the parent opioid begins to decline. At a suprapharmacological doses the bioavailability of the opioid conjugate is substantially decreased as compared to the parent opioid. The relative decrease in bioavailability at higher doses abates the euphoria obtained when doses of the opioid conjugate are taken above those of the intended prescription. This in turn diminishes the abuse potential, whether unintended or intentionally sought.

Persons that abuse opioids such as hydrocodone or oxycodone commonly seek to increase their euphoria by snorting or injecting the drugs. These routes of administration increase the rate and extent of drug absorption and provide a faster, nearly instantaneous, effect. This increases the amount of drug that reaches the central nervous system where it has its effect. In a particular embodiment of the invention the bioavailability of the covalently modified opioid is substantially decreased by the intranasal and intravenous routes as compared to the parent opioid compound. Thus the illicit practice of snorting and shooting the drug loses its advantage.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present invention.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regimens for administration of the active agent are well-known in the relevant art and would be understood to be encompassed by the skilled artisan once provided the teachings disclosed herein.

The amount of the compound of Formula I or II administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to about 7 g/day, preferably about 0.1 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day.

As used herein, the term “combination therapy” refers to the administration of a compound of Formula I or II together with an at least one additional pharmaceutical or medicinal agent, either sequentially or simultaneously.

The present invention includes the use of a combination of a compound as provided in Formula I or II and one or more additional pharmaceutically active agent(s). If a combination of active agents is administered, then they may be administered sequentially or simultaneously, in separate dosage forms or combined in a single dosage form. Accordingly, the present invention also includes pharmaceutical compositions comprising an amount of: (a) a first agent comprising a compound of Formula I or II or a pharmaceutically acceptable salt of the compound; (b) a second pharmaceutically active agent; and (c) a pharmaceutically acceptable carrier, vehicle or diluent.

Various pharmaceutically active agents may be selected for use in conjunction with the compounds of Formula I or II, depending on the disease, disorder, or condition to be treated.

### DETAILED DESCRIPTION

Compounds of the Formula I and II may be prepared from the starting material metopon using methods known to those skilled in the art. See for example U.S. Pat. No. 2,178,010, issued Oct. 31, 1939. Numerous other methods are known to secure the metopon base structure including Gates et. al., J. Org. Chem., 47, 1347-1349 (1982).

In general the compounds of this invention may be made by processes which include processes analogous to those known in the chemical arts, particularly in light of the description contained herein. Prodrugs of the invention may be produced by replacing one of the three appropriate functionalities present in the compounds of Formula I or II with certain moieties known to those skilled in the art as ‘pro-moieties’ as described, for example, in Design of Prodruqs by H. Bundgaard (Elsevier, 1985). See also Pro-drugs as Novel Delivery Systems, Vol. 14, ACS Symposium Series (T. Higuchi and W. Stella) and Bioreversible Carriers in Drug Design, Pergamon Press, 1987 (Ed. E. B. Roche, American Pharmaceutical Association).

As an initial note, in the preparation of the Formula I or II compounds it is noted that some of the preparation methods useful for the preparation of the compounds described herein may require protection of remote functionality (e.g., primary amine, secondary amine, carboxyl in Formula I precursors). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see T. W. Greene, Protective Groups in Organic Synthesis, John Wiley & Sons, New York, 1991.

Compounds of Formula I and II that have chiral centers may exist as stereoisomers, such as racemates, enantiomers, or diastereomers. Conventional techniques for the preparation/isolation of individual enantiomers include chiral synthesis from a suitable optically pure precursor or resolution of the racemate using, for example, chiral high pressure liquid chromatography (HPLC). Alternatively, the racemate (or a racemic precursor) may be reacted with a suitable optically active compound, for example, an alcohol, or, in the case where the compound contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to one skilled in the art. Chiral compounds of Formula I (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture. Stereoisomeric conglomerates may be separated by conventional techniques known to those skilled in the art. See, e.g. “Stereochemistry of Organic Compounds” by E. L. Eliel (Wiley, New York, 1994), the disclosure of which is incorporated herein by reference in its entirety.

Where a compound of Formula I or II contains an alkenyl or alkenylene group, geometric cis/trans (or Z/E) isomers are possible. Cis/trans isomers may be separated by conventional techniques well known to those skilled in the art, for example, chromatography and fractional crystallization. Salts of the present invention can be prepared according to methods known to those of skill in the art.

The compounds of Formula I that are basic in nature are capable of forming a wide variety of salts with various inorganic and organic acids. Although such salts must be pharmaceutically acceptable for administration to animals, it is often desirable in practice to initially isolate the compound of the present invention from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention can be prepared by treating the base compound with a substantially equivalent amount of the selected mineral or organic acid in an aqueous solvent medium or in a suitable organic solvent, such as methanol or ethanol. Upon evaporation of the solvent, the desired solid salt is obtained. The desired acid salt can also be precipitated from a solution of the free base in an organic solvent by adding an appropriate mineral or organic acid to the solution.

Those compounds of Formula I or II that are acidic in nature are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds of Formula I or II. These salts may be prepared by any suitable method, for example, treatment of the free acid with an inorganic or organic base, such as an amine (primary, secondary or tertiary), an alkali metal hydroxide or alkaline earth metal hydroxide, or the like. These salts can also be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

If the inventive compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

Pharmaceutically acceptable salts of compounds of formula I or II may be prepared by one or more of three methods:

(i) by reacting the compound of formula I or II with the desired acid or base;

(ii) by removing an acid- or base-labile protecting group from a suitable precursor of the compound of formula I or II or by ring-opening a suitable cyclic precursor, for example, a lactone or lactam, using the desired acid or base; or

(iii) by converting one salt of the compound of formula I or II to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

Polymorphs can be prepared according to techniques well-known to those skilled in the art.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula I contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

The invention also includes isotopically-labeled compounds of Formula I or II, wherein one or more atoms is replaced by an atom having the same atomic number, but an atomic mass or mass number different from the atomic mass or mass number usually found in nature. Isotopically-labeled compounds of Formula I or II can generally be prepared by conventional techniques known to those skilled in the art or by processes analogous to those described herein, using an appropriate isotopically-labeled reagent in place of the non-labeled reagent otherwise employed.

Compounds of the invention intended for pharmaceutical use may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

They may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

The compounds of the invention may be administered orally. Oral administration may involve swallowing, so that the compound enters the gastrointestinal tract, and/or buccal, lingual, or sublingual administration by which the compound enters the blood stream directly from the mouth.

Formulations suitable for oral administration include solid, semi-solid and liquid systems such as tablets; soft or hard capsules containing multi- or nano-particulates, liquids, or powders; lozenges (including liquid-filled); chews; gels; fast dispersing dosage forms; films; ovules; sprays; and buccal/mucoadhesive patches.

Liquid formulations include suspensions, solutions, syrups and elixirs. Such formulations may be employed as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a compound of formula I, a film-forming polymer, a binder, a solvent, a humectant, a plasticiser, a stabiliser or emulsifier, a viscosity-modifying agent and a solvent. Some components of the formulation may perform more than one function.

The compound of formula I or II may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, the compound of formula I or II may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Pharmaceutical Technology On-line, 25(2), 1-14, by Verma et al (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

The compounds of the invention can also be formulated as Drug-cyclodextrin complexes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

Since the present invention has an aspect that relates to the treatment of the disease/conditions described herein with a combination of active ingredients which may be administered separately, the invention also relates to combining separate pharmaceutical compositions in kit form. The kit comprises two separate pharmaceutical compositions: a compound of Formula I or II or a salt of such compound and a second compound as described above. The kit comprises means for containing the separate compositions such as a container, a divided bottle or a divided foil packet. Typically the kit comprises directions for the administration of the separate components.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

All publications, including but not limited to, issued patents, patent applications, and journal articles, cited in this application are each herein incorporated by reference in their entirety.

Although the invention has been described above with reference to the disclosed embodiments, those skilled in the art will readily appreciate that the specific experiments detailed below are only illustrative of the invention. It should be understood that various modifications can be made without departing from the spirit of the invention. Accordingly, the invention is limited only by the claims.

1. A compound of Formula 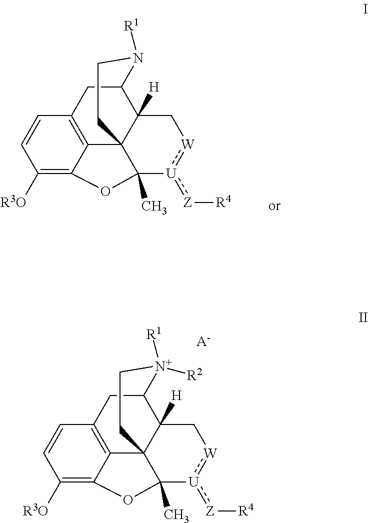
R¹ is alkyl, preferably methyl, ethyl, isopropyl or cyclopropyl;
R² is O⁻, CHR⁵OPO₃X₂, CHR⁵OPO₃H₂, CHR⁵OPO(OCHR⁵OCO₂R⁶)₂, CHR⁵OPO(OCHR⁵OCOR⁶)₂, CHR⁵OCO₂R⁶ (wherein R⁶ is other than H), or CHR⁵OCOR⁶;
R³ is H (except that R¹ cannot be CH₃ in Formula I when U—Z is carbonyl); alkyl preferably CH₃ (except that R¹ cannot be CH₃ in Formula I when U—Z is carbonyl), or R⁷;
R⁴ is absent when U—Z is carbonyl, or
Is COR⁷ when U—W is a C—C double bond and U—Z is a single C—O bond, or
Is H, alkyl, OH or O-alkyl when Z is N and U—Z is carbon-nitrogen double bond and U—W is a single bond;
R⁵ is H or alkyl (preferably methyl, ethyl, isopropyl or cyclopropyl);
R⁶ is H, alkyl, cycloalkyl, phenylalkyl, heteroalkyl, heterocycloalkyl, aryl, heteroaryl, or a sugar such as galactose;
R⁷ wherever it occurs is independently selected from alkyl (with the proviso that alkyl cannot be CH₃ in formula I when U—Z is carbonyl), cycloalkyl, phenylalkyl, heteroalkyl, heterocycloalkyl, CHR⁵OCO₂R⁶, CHR⁵OCOR⁶, CHR⁵₂, (CH₂)ₙCO₂X, (CH₂)ₙCO₂H, (CH₂)ₙNR⁶₂, CHR⁵OPO₃H, CHR⁵OPO₃X, CHR⁶NHR⁶, alkylCO₂X, alkylCO₂H, CHR⁵OCOR⁶, CHR⁵OCO₂R⁶ (wherein R⁶ is other than H), CHR⁵OPO(OCHR⁵OCOR⁶)₂, CHR⁵OCOCHR⁵NH₂, COR⁸, COCHCHCO₂H, COCHCHCO₂X, COCH₂CH₂SO₃X, CO(CH₂)ₙCONHCO(CH₂)ₙCONHCH₂CH₂SO₃X, CONMeCH₂CF₂CH₂NH₂, COCHR⁵NR⁶R⁶, CO₂R⁶, COR⁶, CONR⁶₂, COCOR⁶, COCOOR⁶, COSR⁶, CSR⁶, CSOR⁶, SO₂NR⁵R⁶, SO₂OR⁶, SO₂R⁶, CONHCHR⁵CO₂CH₂CHNH₂CO₂H, CONHCHR⁵CO₂CH₂CHNH₂CO₂X, CONHCHR⁵CO₂CH₂CHNH₂CO₂H, CHR⁵OPO(OCHR⁵OCO₂R⁶)₂, CHR⁵OPO(OCHR⁵OCOR⁶)₂, CON(CH₃)(CH₂)₂NHCOCH(CH₃)CH2CH2CH2(NCOCH₃)CNHNH₂, CO(CH₂)₃CONHCHR⁵CO₂H, PO₃X₂, PO₃H₂, PO₃R⁶₂, COCHR⁹(NCOCHR⁹)ₙNH₂, 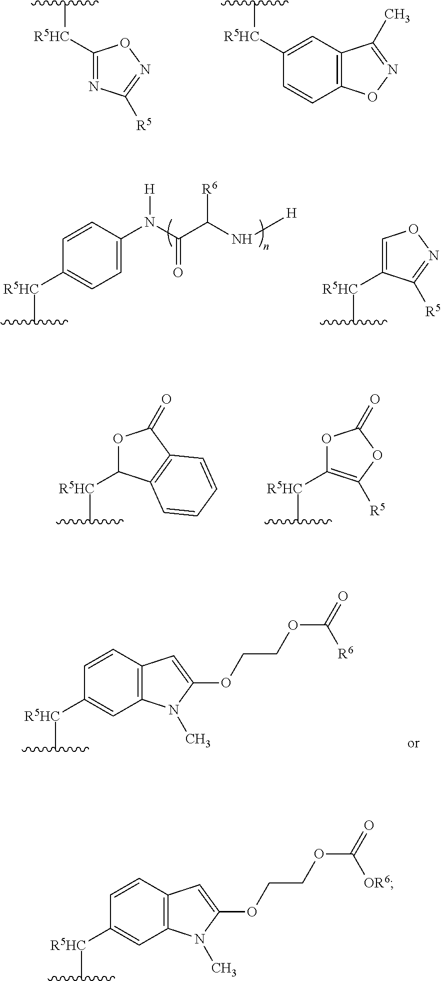
R⁸ is alkyl, cycloalkyl, phenylalkyl, heteroalkyl, heterocycloalkyl, aryl, heteroaryl, 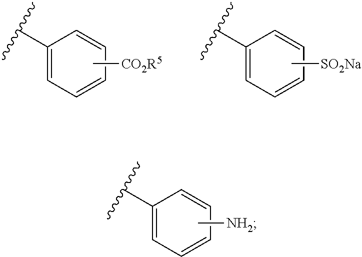
R⁹ is a 1 to 6, more preferably 1 to 4, natural, synthetic, racemic, L-, or D-amino acid group;
A is an anion selected from the group consisting of: Br⁻, Cl⁻, I⁻, R⁷CO₂⁻ (lactate, acetate, tartrate, valerate), H₂PO₄⁻, NO₃⁻, and R⁶SO₃⁻
X is a cation independently selected from the group consisting of: Li⁺, Na⁺, K⁺, Mg⁺, ⁺NH₃R⁷, ⁺NH₂R⁷₂, ⁺NHR⁷₃, ⁺NR⁷₄, and ⁺NH₃(CH₂)ₙOH;
U is C;
W is CH₂ or CH;
Z═O; NH; NOH; NCH₃; NOR⁵;
Each n is an integer independently selected from 1 to 6.

2. A compound of Formula I 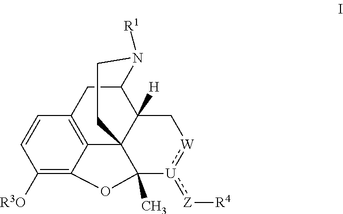
R¹ is methyl, ethyl, isopropyl or cyclopropyl;
R² is O⁻, CHR⁵OPO₃X₂, CHR⁵OPO₃H₂, CHR⁵OPO(OCHR⁵OCO₂R⁶)₂, CHR⁵OPO(OCHR⁵OCOR⁶)₂, CHR⁵OCO₂R⁶ (wherein R⁶ is other than H), or CHR⁵OCOR⁶;
R³ is H (except that R¹ can not be CH₃ in Formula I when U—Z is carbonyl); alkyl preferably CH₃ (except that R¹ can not be CH₃ in Formula I when U—Z is carbonyl), or R⁷;
R⁴ is absent when U—Z is carbonyl, or
Is COR⁷ when U—W is C—C double bond and U—Z is single C—O bond, or
Is H, alkyl, OH or O-alkyl when Z is N and U—Z is carbon-nitrogen double bond and U—W is a single bond;
R⁵ is H or alkyl (preferably methyl, ethyl, isopropyl or cyclopropyl);
R⁶ is H, alkyl, cycloalkyl, phenylalkyl, heteroalkyl, heterocycloalkyl, aryl, heteroaryl, or a sugar such as galactose;
R⁷ wherever it occurs is independently selected from alkyl (with the proviso that alkyl can not be CH₃ in formula I when U—Z is carbonyl), cycloalkyl, phenylalkyl, heteroalkyl, heterocycloalkyl, CHR⁵OCO₂R⁶, CHR⁵OCOR⁶, CHR⁵₂, (CH₂)ₙCO₂X, (CH₂)ₙCO₂H, (CH₂)ₙNR⁶₂, CHR⁵OPO₃H, CHR⁵OPO₃X, CHR⁶NHR⁶, alkylCO₂X, alkylCO₂H, CHR⁵OCOR⁶, CHR⁵OCO₂R⁶ (wherein R⁶ is other than H), CHR⁵OPO(OCHR⁵OCOR⁶)₂, CHR⁵OCOCHR⁵NH₂, COR⁸, COCHCHCO₂H, COCHCHCO₂X, COCH₂CH₂SO₃X, CO(CH₂)ₙCONHCO(CH₂)ₙCONHCH₂CH₂SO₃X, CONMeCH₂CF₂CH₂NH₂, COCHR⁵NR⁶R⁶, CO₂R⁶, COR⁶, CONR⁶₂, COCOR⁶, COCOOR⁶, COSR⁶, CSR⁶, CSOR⁶, SO₂NR⁵R⁶, SO₂OR⁶, SO₂R⁶, CONHCHR⁵CO₂CH₂CHNH₂CO₂H, CONHCHR⁵CO₂CH₂CHNH₂CO₂X, CONHCHR⁵CO₂CH₂CHNH₂CO₂H, CHR⁵OPO(OCHR⁵OCO₂R⁶)₂, CHR⁵OPO(OCHR⁵OCOR⁶)₂, CON(CH₃)(CH₂)₂NHCOCH(CH₃)CH2CH2CH2(NCOCH₃)CNHNH₂, CO(CH₂)₃CONHCHR⁵CO₂H, PO₃X₂, PO₃H₂, PO₃R⁶₂, COCHR⁹(NCOCHR⁹)ₙNH₂, 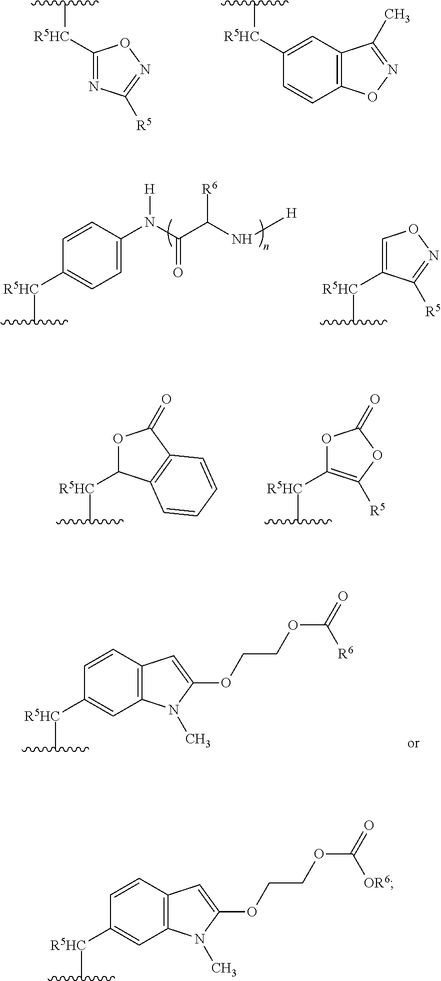
R⁸ is alkyl, cycloalkyl, phenylalkyl, heteroalkyl, heterocycloalkyl, aryl, heteroaryl, 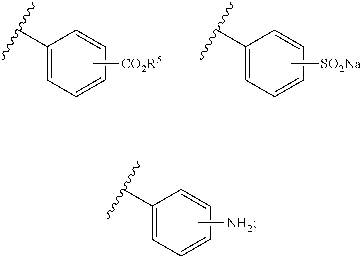
R⁹ is a 1 to 6, more preferably 1 to 4, natural, synthetic, racemic, L-, or D-amino acid group;
A is an anion selected from the group consisting of: Br⁻, Cl⁻, I⁻, R⁷CO₂⁻ (lactate, acetate, tartrate, valerate), H₂PO₄⁻, NO₃⁻, and R⁶SO₃⁻
X is a cation independently selected from the group consisting of: Li⁺, Na⁺, K⁺, Mg⁺, ⁺NH₃R⁷, ⁺NH₂R⁷₂, ⁺NHR⁷₃, ⁺NR⁷₄, and ⁺NH₃(CH₂)ₙOH;
U is C;
W is CH₂ or CH;
Z═O; NH; NOH; NMe; NOR⁵;
Each n is an integer independently selected from 1 to 6.

3. A compound according to claim 2 wherein R³ is H, U—Z is carbonyl and R¹ is ethyl, isopropyl or cyclopropyl.

4. A compound according to claim 2 wherein R³ is alkyl, preferably CH₃, U—Z is carbonyl and R¹ is ethyl, isopropyl or cyclopropyl.

5. A compound according to claim 2 wherein R³ is H, U—W is a C—C double bond, U—Z is a single C—O bond, R⁴ is COR⁷, and R¹ is methyl, ethyl, isopropyl or cyclopropyl; and
R⁷ is selected from alkyl, cycloalkyl, phenylalkyl, heteroalkyl, heterocycloalkyl, CHR⁵OCO₂R⁶, CHR⁵OCOR⁶, CHR⁵₂, (CH₂)ₙCO₂X, (CH₂)ₙCO₂H, (CH₂)ₙNR⁶₂, CHR⁵OPO₃H, CHR⁵OPO₃X, CHR⁶NHR⁶, alkylCO₂X, alkylCO₂H, CHR⁵OCOR⁶, CHR⁵OCO₂R⁶ (wherein R⁶ is other than H), CHR⁵OPO(OCHR⁵OCOR⁶)₂, CHR⁵OCOCHR⁵NH₂, COR⁸, COCHCHCO₂H, COCHCHCO₂X, COCH₂CH₂SO₃X, CO(CH₂)ₙCONHCO(CH₂)ₙCONHCH₂CH₂SO₃X, CONMeCH₂CF₂CH₂NH₂, COCHR⁵NR⁶R⁶, CO₂R⁶, COR⁶, CONR⁶₂, COCOR⁶, COCOOR⁶, COSR⁶, CSR⁶, CSOR⁶, SO₂NR⁵R⁶, SO₂OR⁶, SO₂R⁶, CONHCHR⁵CO₂CH₂CHNH₂CO₂H, CONHCHR⁵CO₂CH₂CHNH₂CO₂X, CONHCHR⁵CO₂CH₂CHNH₂CO₂H, CHR⁵OPO(OCHR⁵OCO₂R⁶)₂, CHR⁵OPO(OCHR⁵OCOR⁶)₂, CON(CH₃)(CH₂)₂NHCOCH(CH₃)CH2CH2CH2(NCOCH₃)CNHNH₂, CO(CH₂)₃CONHCHR⁵CO₂H, PO₃X₂, PO₃H₂, PO₃R⁶₂, COCHR⁹(NCOCHR⁹)ₙNH₂,

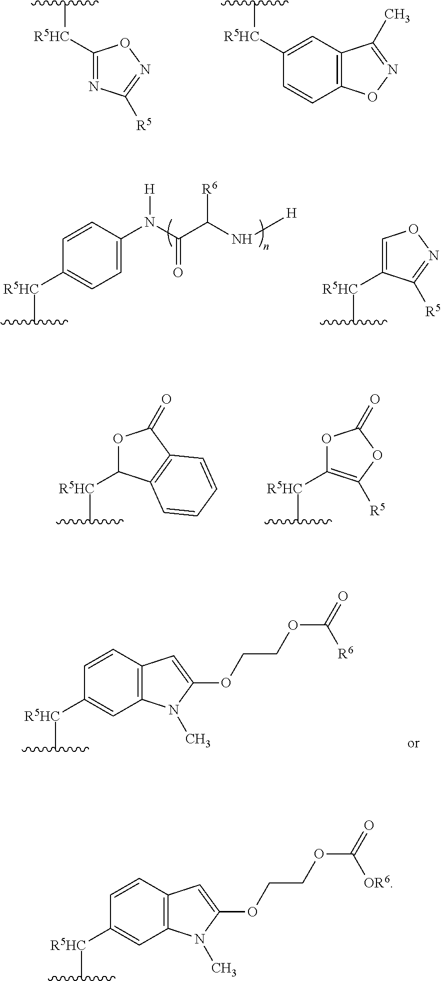

6. A compound according to claim 2 wherein R³ is R⁷.

7. A compound according to claim 6 wherein R³ is R⁷; U—Z is carbonyl and R¹ is ethyl, isopropyl or cyclopropyl.

8. A compound according to claim 6 wherein R³ is R⁷; U—W is a C—C double bond, U—Z is a single C—O bond, R⁴ is COR⁷.

9. A compound of Formula II 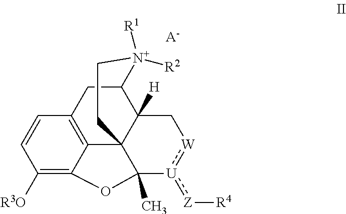
R¹ is alkyl, preferably methyl, ethyl, isopropyl or cyclopropyl;
R² is O⁻, CHR⁵OPO₃X₂, CHR⁵OPO₃H₂, CHR⁵OPO(OCHR⁵OCO₂R⁶)₂, CHR⁵OPO(OCHR⁵OCOR⁶)₂, CHR⁵OCO₂R⁶ (wherein R⁶ is other than H), or CHR⁵OCOR⁶;
R³ is H, alkyl (preferably CH₃), or R⁷;
R⁴ is absent when U—Z is carbonyl, or
Is COR⁷ when U—W═C—C double bond and U—Z=single C—O bond, or
Is H, alkyl, OH or O-alkyl when Z is N and U—Z is carbon-nitrogen double bond and U—W is a single bond;
R⁵ is H or alkyl (preferably methyl, ethyl, isopropyl or cyclopropyl);
R⁶ is H, alkyl, cycloalkyl, phenylalkyl, heteroalkyl, heterocycloalkyl, aryl, heteroaryl, or a sugar such as galactose;
R⁷ wherever it occurs is independently selected from alkyl, cycloalkyl, phenylalkyl, heteroalkyl, heterocycloalkyl, CHR⁵OCO₂R⁶, CHR⁵OCOR⁶, CHR⁵₂, (CH₂)ₙCO₂X, (CH₂)ₙCO₂H, (CH₂)ₙNR⁶₂, CHR⁵OPO₃H, CHR⁵OPO₃X, CHR⁶NHR⁶, alkylCO₂X, alkylCO₂H, CHR⁵OCOR⁶, CHR⁵OCO₂R⁶ (wherein R⁶ is other than H), CHR⁵OPO(OCHR⁵OCOR⁶)₂, CHR⁵OCOCHR⁵NH₂, COR⁸, COCHCHCO₂H, COCHCHCO₂X, COCH₂CH₂SO₃X, CO(CH₂)ₙCONHCO(CH₂)ₙCONHCH₂CH₂SO₃X, CONMeCH₂CF₂CH₂NH₂, COCHR⁵NR⁶R⁶, CO₂R⁶, COR⁶, CONR⁶₂, COCOR⁶, COCOOR⁶, COSR⁶, CSR⁶, CSOR⁶, SO₂NR⁵R⁶, SO₂OR⁶, SO₂R⁶, CONHCHR⁵CO₂CH₂CHNH₂CO₂H, CONHCHR⁵CO₂CH₂CHNH₂CO₂X, CONHCHR⁵CO₂CH₂CHNH₂CO₂H, CHR⁵OPO(OCHR⁵OCO₂R⁶)₂, CHR⁵OPO(OCHR⁵OCOR⁶)₂, CON(CH₃)(CH₂)₂NHCOCH(CH₃)CH2CH2CH2(NCOCH₃)CNHNH₂, CO(CH₂)₃CONHCHR⁵CO₂H, PO₃X₂, PO₃H₂, PO₃R⁶₂, COCHR⁹(NCOCHR⁹)ₙNH₂, 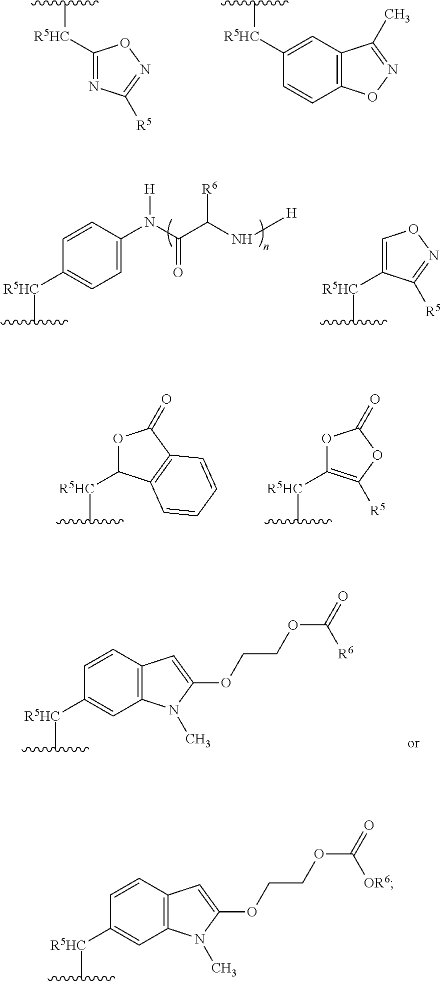
R⁸ is alkyl, cycloalkyl, phenylalkyl, heteroalkyl, heterocycloalkyl, aryl, heteroaryl, 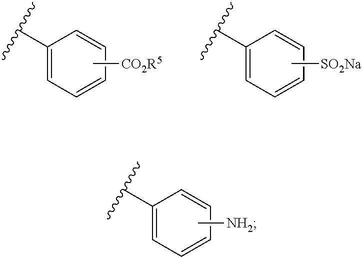
R⁹ is a 1 to 6, more preferably 1 to 4, natural, synthetic, racemic, L-, or D-amino acid group;
A is an anion selected from the group consisting of: Br⁻, Cl⁻, I⁻, R⁷CO₂⁻ (lactate, acetate, tartrate, valerate), H₂PO₄⁻, NO₃⁻, and R⁶SO₃⁻
X is a cation independently selected from the group consisting of: Li⁺, Na⁺, K⁺, Mg⁺, ⁺NH₃R⁷, ⁺NH₂R⁷₂, ⁺NHR⁷₃, ⁺NR⁷₄, and ⁺NH₃(CH₂)ₙOH;
U is C;
W is CH₂ or CH;
Z═O; NH; NOH; NMe; NOR⁵;
Each n is an integer independently selected from 1 to 6.

10. A compound according to claim 9 wherein R⁴ is absent and U—Z is carbonyl.

11. A compound according to claim 10 wherein R³ is H or alkyl.

12. A compound according to claim 10 wherein R³ is R⁷.

13. A compound according to claim 9 wherein U—W is a C—C double bond; U—Z is a single C—O bond and R⁴ is COR⁷.

14. A compound according to claim 13 wherein R³ is H or alkyl.

15. A compound according to claim 9 wherein Z is N, U—Z is a carbon-nitrogen double bond, U—W is a C—C single bond; and R⁴ is H, alkyl, OH or O-alkyl.

16. A compound according to claim 15 wherein R³ is H or alkyl.

17. The according to claim 15 wherein said compound comprises a composition in tablet, capsule, oral solution, or oral suspension dosage form.

18. A method of treating acute or chronic pain comprising administering to a patient the composition of claim 17.

19. A method of treating acute or chronic pain comprising administering to a patient the compound of claim 1.

20. The method of claim 18 wherein said compound is formulated into a tablet, a capsule, an oral solution, or an oral suspension.

21. The composition of claim 17 wherein said composition is in tablet or capsule dosage form.

22. The method of claim 20 wherein said compound is in tablet form.

23. The method of claim 20 wherein said compound is in capsule form.
